(19) United States
(12) Patent Application Publication
(10) Pub. No.: US 2017/0181490 A1
(43) Pub. Date: Jun. 29, 2017

(54) SYSTEMS AND METHODS FOR POSITIONING EYEWEAR

(71) Applicant: Joshua J. Buckner-Johnson, Bothell, WA (US)

(72) Inventor: Joshua J. Buckner-Johnson, Bothell, WA (US)

(21) Appl. No.: 14/983,340

(22) Filed: Dec. 29, 2015

(51) Int. Cl. A42B 3/18; A61F 9/02

(52) U.S. Cl. CPC A42B 3/185; A61F 9/027

(57) ABSTRACT

A device with a stationary portion and a rotatable portion, wherein the rotatable portion is lifted to form a substantially L-shaped bracket. Device is configured to be disposed on the forehead of the wearer, preferably coupled with protective headgear. When the rotatable portion is lifted, the bracket is configured for receiving an edge of eyewear. When the eyewear is pulled upward and back due to forces exerted by straps disposed around a user's head, the bracket exerts an equal and opposite force on the edge of the eyewear, holding the eyewear in place. Bracket may include grips for easier use in inclement weather or when the user's hands are otherwise encumbered.

### FIELD OF THE INVENTION

This invention relates generally to eyewear, and, more specifically, to systems and methods for positioning eyewear.

### BACKGROUND OF THE INVENTION

Many activities require that a user don and remove protective gear repeatedly throughout the course of the activity. When combined with other protective gear, donning and removing eyewear can be cumbersome and frustrating for the wearer. This is especially true if the user is also wearing a helmet, hardhat, or other head protection. The present invention aims to overcome these burdens, helping a user to quickly place and remove eyewear, even while wearing protective head gear, gloves, mittens, and other protective clothing.

### SUMMARY OF THE INVENTION

This invention relates generally to eyewear, and, more specifically, to systems and methods for positioning eyewear.

A device for positioning eyewear, the device comprising: a stationary portion with a top and a bottom; a rotatable portion with a top and a bottom, the rotatable portion coupled with at least one fastener, wherein the rotatable portion rotates about the at least one fastener. In some embodiments, the device may further comprise: an upper edge disposed across the top of the stationary portion; and a lower edge disposed across the bottom of the stationary portion, wherein the rotatable portion fits substantially within a boundary formed by the upper edge and the lower edge. In some embodiments, the upper edge extends onto at least one side of the stationary portion. In some embodiments, the lower edge extends onto at least one side of the stationary portion. In other embodiments, the upper edge extends across the top of the stationary portion, and wherein the lower edge extends across the bottom of the stationary portion such that the upper edge and the lower edge form at least one gap on at least one side of the stationary portion. In some embodiments, the rotatable portion is disposed such that it fits within the upper edge and the lower edge of the stationary portion. In some embodiments, the rotatable portion may further comprise: at least one grip, the at least one grip configured to fit within the at least one gap on the at least one side of the stationary portion. In some embodiments, the upper edge and the lower edge form at least one gap on at least two sides of the stationary portion. In some embodiments, the rotatable portion further comprises: at least one grip, the at least one grip configured to fit within at least one gap on the at least two sides of the stationary portion.

In some embodiments, an outer edge of the rotatable portion is substantially flush with the upper edge and lower edge of the stationary portion. In some embodiments, the stationary portion further comprises: at least one front portion; and at least one rear portion. In some embodiments, the at least one rear portion is curved such that it substantially follows the contour of at least one protective equipment. In some further embodiments, the at least one rear portion includes at least one adhesive surface. In some embodiments, the rotatable portion further comprises: at least one front portion; and at least one rear portion. In some embodiments, the at least one fastener includes at least one spring element.

The present disclosure also includes a method of holding eyewear in place. In preferred embodiments, the method includes: coupling a system for holding eyewear to at least one protective headgear; disposing at least one strap of at least one eyewear around the at least one headgear; lifting at least one rotatable portion of the system for holding eyewear; and disposing an upper edge of the at least one eyewear below the at least one rotatable portion of the system such that the eyewear exerts an upward force on the at least one rotatable portion.

In addition to the foregoing, various other methods, systems and/or program product embodiments are set forth and described in the teachings such as the text (e.g., claims, drawings and/or the detailed description) and/or drawings of the present disclosure.

The foregoing is a summary and thus contains, by necessity, simplifications, generalizations and omissions of detail; consequently, those skilled in the art will appreciate that the summary is illustrative only and is NOT intended to be in any way limiting. Other aspects, embodiments, features and advantages of the device and/or processes and/or other subject matter described herein will become apparent in the teachings set forth herein.

### BRIEF DESCRIPTION OF THE DRAWINGS

Certain embodiments of the present invention are described in detail below with reference to the following drawings:

FIG. 1 is an isometric view of one embodiment the system for positioning eyewear in the open position.

FIG. 2 is a front isometric view of one embodiment of the system for positioning eyewear in the closed position.

FIG. 3 is a rear isometric view thereof.

FIG. 4 is a detailed view thereof

FIG. 5 is an exploded view thereof

FIG. 6 is an environmental view of one embodiment of the system for positioning eyewear in the closed position.

FIG. 7 is an environmental view of one embodiment of the system for positioning eyewear in the open position.

### DETAILED DESCRIPTION

This invention relates generally to eyewear, and, more specifically, to systems and methods for positioning eyewear.

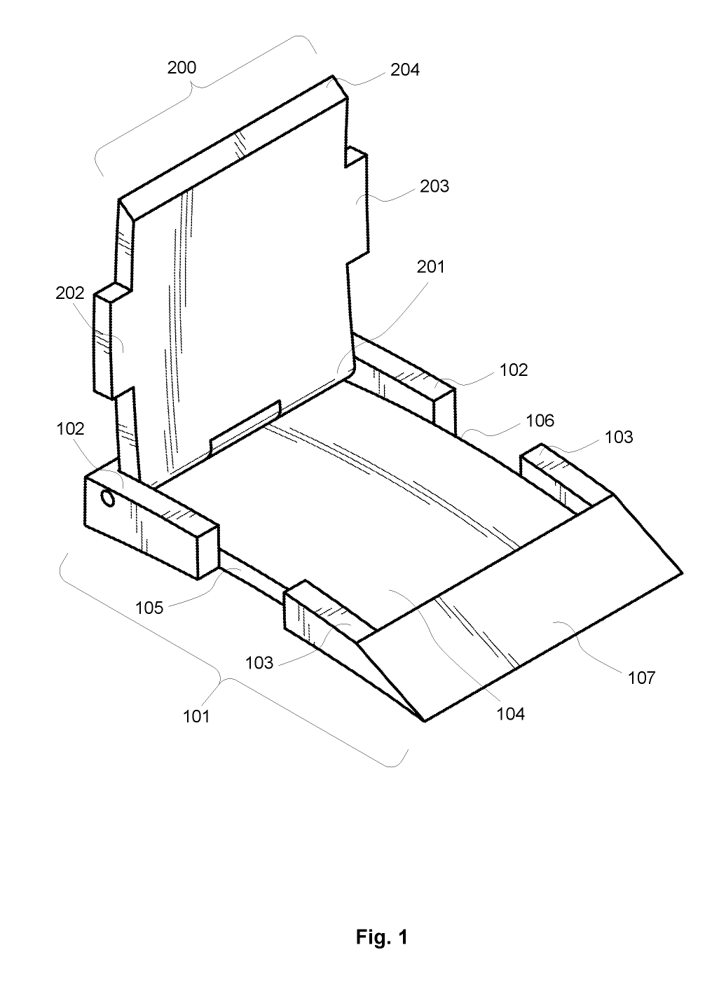

Specific details of certain embodiments of the invention are set forth in the following description and in FIGS. 1-7 to provide a thorough understanding of such embodiments. The present invention may have additional embodiments, may be practiced without one or more of the details described for any particular described embodiment, or may have any detail described for one particular embodiment practiced with any other detail described for another embodiment.

Importantly, a grouping of inventive aspects in any particular “embodiment” within this detailed description, and/or a grouping of limitations in the claims presented herein, is not intended to be a limiting disclosure of those particular aspects and/or limitations to that particular embodiment and/or claim. The inventive entity presenting this disclosure fully intends that any disclosed aspect of any embodiment in the detailed description and/or any claim limitation ever presented relative to the instant disclosure and/or any continuing application claiming priority from the instant application (e.g. continuation, continuation-in-part, and/or divisional applications) may be practiced with any other disclosed aspect of any embodiment in the detailed description and/or any claim limitation. Claimed combinations which draw from different embodiments and/or originally-presented claims are fully within the possession of the inventive entity at the time the instant disclosure is being filed. Any future claim comprising any combination of limitations, each such limitation being herein disclosed and therefore having support in the original claims or in the specification as originally filed (or that of any continuing application claiming priority from the instant application), is possessed by the inventive entity at present irrespective of whether such combination is described in the instant specification because all such combinations are viewed by the inventive entity as currently operable without undue experimentation given the disclosure herein and therefore that any such future claim would not represent new matter.

FIG. 1 an isometric view of one embodiment of the system for positioning eyewear in the open position. The system is comprised essentially of a fixed base 101 and a movable flap 200. In some embodiments, flap 200 sits flush within a recess 104 within the base, and is able to rotate from zero degrees, wherein it is disposed within the recess, to 120 degrees from the base. In preferred embodiments, flap 200 rotates from zero degrees through 90 degrees from base 101. In some embodiments, flap 200 is coupled with base 101 at pivot point 201.

In some embodiments, recess 104 may be formed by upper edge 102 and lower edge 103 on base 101. In some embodiments, upper edge 102 and lower edge 103 may completely encompass recess 104, effectively creating a perimeter around the entire recess. In other embodiments, upper edge 102 and lower edge 103 may be disposed only along the upper and lower portions of the base 101, without extending onto either side of the base. In preferred embodiments, upper edge 102 and lower edge 103 each extend onto both sides of the base 101. In a further embodiment, upper edge 102 does not quite meet lower edge 103, leaving a gap 105 on the left side of the invention and a gap 106 on the right side of the invention. This forms a partial perimeter, allowing a user access to flap 200 via gaps 105 and 106.

In some embodiments, flap 200 may have at least one grip, such as left grip 202. In some embodiments, flap 200 may have two grips, a left grip 202 and a right grip 203. In some embodiments, at least one of grips 202 and 203 may over hang base 101. In other embodiments, at least one of grips 202 and 203 may fit into at least one gap on base 101, such as at least one of gap 105 and 106. One non-limiting purpose of the grips is to allow a user to easily detect where flap 200 is such that the user can reposition the flap for use.

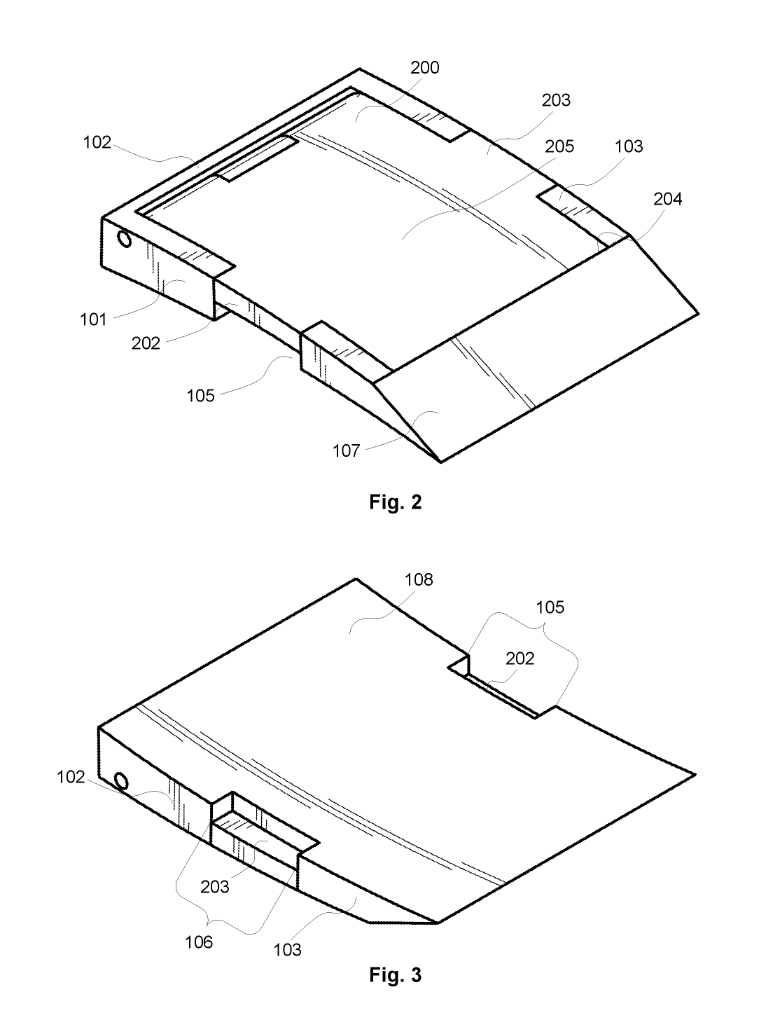

In some embodiments, flap 200 may have a tapered lower edge 204. In some embodiments, tapered lower edge 204 may help flap 200 to sit flush within recess 104. In other embodiments, tapered edge 204 may increase aerodynamic flow when flap 200 is in use. In some embodiments, edge 204 may not be tapered. In other embodiments, edge 204 may be tapered in the opposite orientation from the taper depicted in FIG. 1. In some embodiments, edge 204 may be configured to form a smooth transition with tapered base lip 107. When the device is not in use, it may be present in an environment wherein aerodynamics can play an important role. One function of tapered base lip 107 is to eliminate or drastically reduce fluid resistance over the device when flap 200 is in recess 104. This is better illustrated in FIG. 2.

FIG. 2 is an isometric view of one embodiment of the present invention in the closed position. In one preferred embodiment, the front of the invention is formed when flap 200 is disposed within recess 104, creating a substantially smooth surface area. In some embodiments, this substantially smooth front 205 is slightly curved, generally following the curve of common headgear such as helmets and hardhats. In other embodiments, front 205 may be substantially level or flat, with no appreciable curve.

FIG. 2 depicts how, in some embodiments, grips 202 and 203 fit into gaps 105 and 106, respectively, though gap 106 is not depicted in FIG. 2. FIG. 3 also illustrates this particular embodiment, showing that gaps 105 and 106 are, in at least one embodiment, formed by spacing between upper edge 102 and lower edge 103. In some embodiments, the gaps and grips depicted herein may allow a user to more easily lift flap 200, especially when the user is wearing hand protection such as gloves or mittens, or when the user's hands have reduced function due to exposure.

FIG. 3 also depicts that, in some embodiments, rear 108 of base 101 may be substantially curved. While a curved rear surface 108 is preferred, it would not significantly alter the function of the device if rear surface 108 was substantially level or flat. In some embodiments, base 101, upper edge 102, and lower edge 103 may be separate, coupleable pieces, joined through welding, gluing, riveting, or other means of coupling substantially rigid materials such as metal, hard plastic, or wood. In other embodiments, base 101, upper edge 102, and lower edge 103 may be a single piece of substantially rigid material that has been machined to form the features of the invention, such as the edges and recess 104 formed therefrom. In some embodiments, rear surface 108 may be coupled with a double-sided adhesive material, the adhesive material further coupled with at least one piece of protective headgear. In other embodiments, the system for positioning eyewear 100 may be coupled with at least one piece of protective headgear via a screw, glue, rivet, or other means for joining two rigid or semi-rigid materials. In some embodiments, the system 100 may be word directly on a user's head. In a further embodiment, the system 100 may include one or more straps disposed on at least one side of the system such that the strap could be disposed around a user's head and the system positioned on the user's forehead. In a further embodiment, rear surface 108 may include at least one piece of cushioning or warming material, such as leather, felt, or a pouch containing sodium acetate (hot ice) to keep the user's head warm in cool weather.

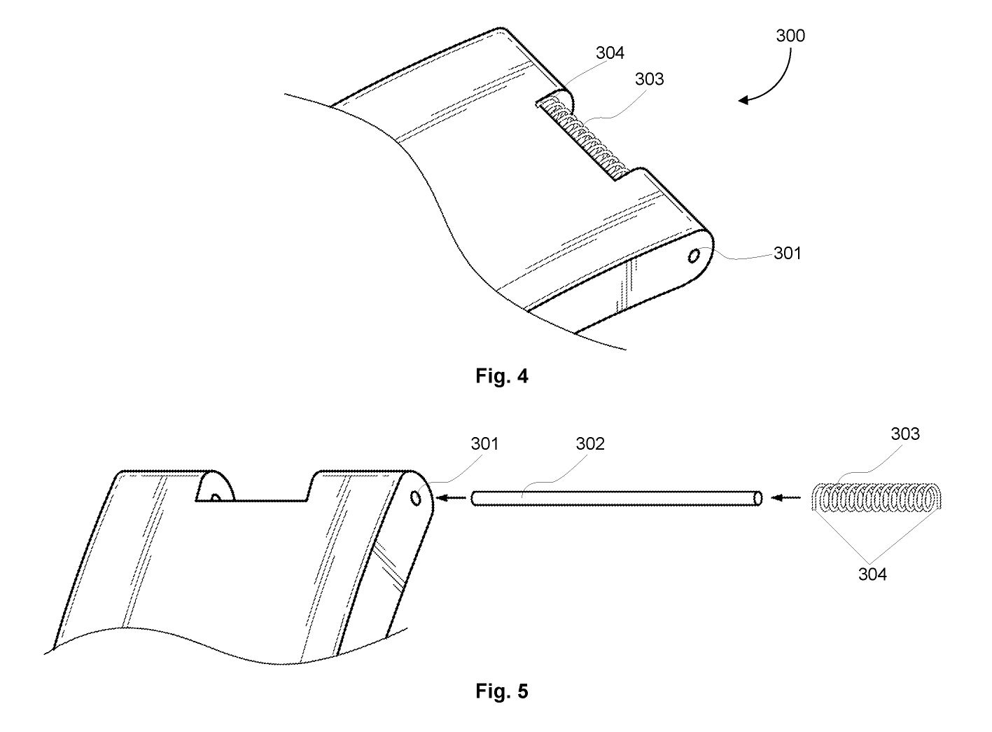

FIGS. 4 and 5 are detailed views of one embodiment of the joint 300 between base 101 and flap 200 of the present invention. FIG. 4 depicts how the joint 300 sits within base 101 when the joint is a spring loaded pivot system. In such an embodiment, a spring 303 and rod 302 (see FIG. 5) may be disposed within hole 301 of base 101. The rod or pin 302 may keep joint 300 in position, while spring 303 allows flap 200 to be lifted. Spring 303 may also be used to return flap 200 to recess 104 (see FIG. 1, 2) when the device is not in use. In some embodiments, spring 303 may have a tab 304 on one or both ends of the spring. FIG. 4 shows an exaggerated view of spring tab 304, depicted outside base 101. Tab 304 may, in some embodiments, be useful in returning flap 200 to recess 104 by causing the spring to be loaded against the direction of lift for flap 200. While FIG. 4 depicts tab 304 outside base 101, in some embodiments, at least one of the tabs may be disposed within an additional hole in either the base, flap 200, or both.

While the spring-rod system depicted here may be a preferred embodiment, it should be understood that any method of pivotally coupling two rigid pieces of material may be employed at joint 300 without significantly altering the function of the device. For instance, a flap of more flexible material could be disposed between base 101 and flap 200. In another non-limiting example, a clock spring or torsion spring could replace coil spring 303. In another non-limiting example, joint 300 may be comprised of only a rod or pin, wherein the user relies on gravity alone to return flap 200 to recess 104.

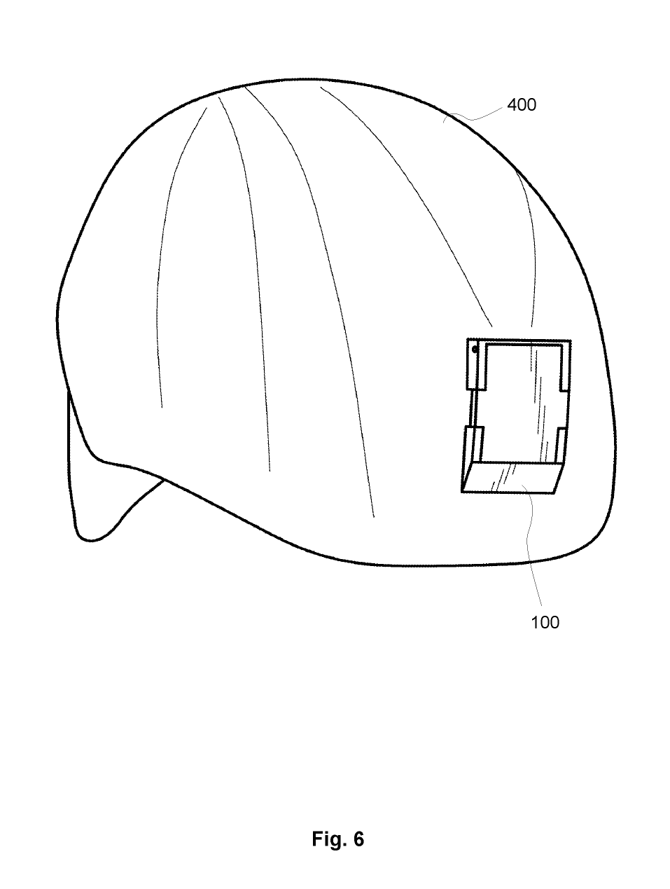

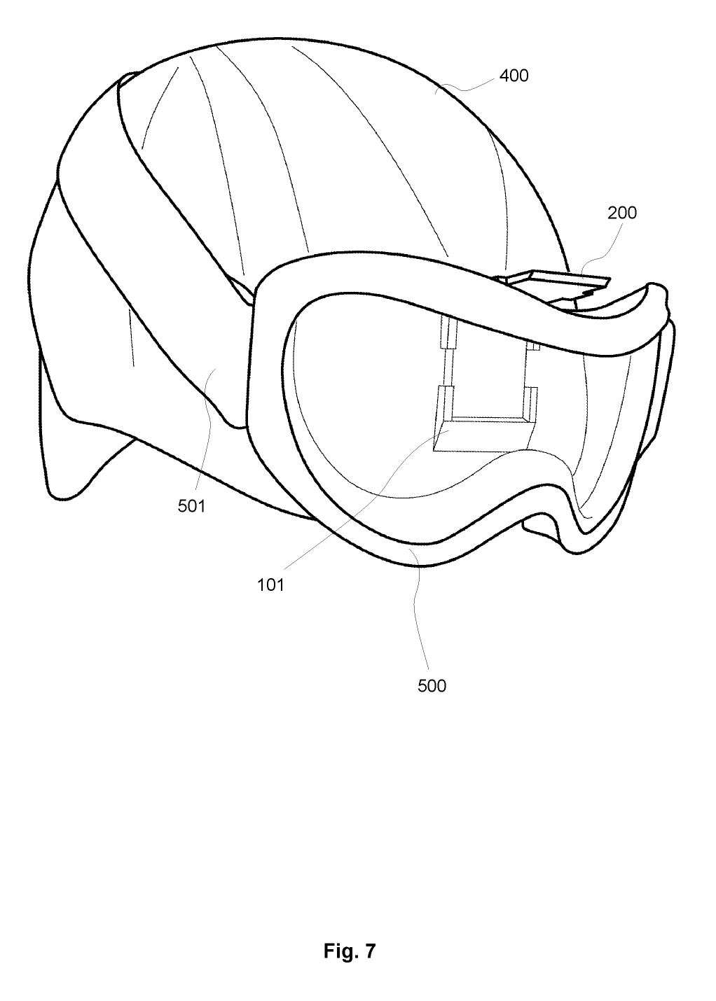

FIGS. 6 and 7 depict a preferred method of use for the present invention. FIG. 6 shows one embodiment of the system for positioning eyewear 100 in the closed position and disposed on a piece of protective headgear 400. This is what the invention would look like when the user is wearing the eyewear over his or her eyes. FIG. 7 shows one embodiment of the present invention when the user is not wearing the eyewear over his or her eyes. Here it can be seen that base 101 is disposed on headgear 400, and flap 200 is raised to approximately 90 degrees to base 101. Eyewear 500 is resting on the surface of headgear 400, and strap 501 is disposed around the headgear. It should be noted that while many pieces of protective headgear have loops or snaps or other means by which a user can removably couple eyewear straps with the rear of the headgear, such a means of coupling is not necessary for the function of the present invention. When strap 501 is disposed around headgear 400, it creates an upward and rearward force on eyewear 500. When flap 200 is lifted and eyewear 500 is placed under it, the flap exerts an equal and opposite force downward on the eyewear, holding the eyewear in place. It should be noted that while ski helmets and goggles are depicted in FIGS. 6 and 7, this method of use would also apply to hardhats and safety glasses with straps, as well as other protective head and eye wear. As noted above, the system disclosed herein could also be used with straps, for example, and disposed directly on a user's forehead.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of this subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

While preferred and alternative embodiments of the invention have been illustrated and described, as noted above, many changes can be made without departing from the spirit and scope of the invention. Accordingly, the scope of the invention is not limited by the disclosure of these preferred and alternate embodiments. Instead, the invention should be determined entirely by reference to the claims that follow.

What is claimed is:

1. A system for positioning eyewear, the system comprising:
a stationary portion with a top and a bottom;
a rotatable portion with a top and a bottom, the rotatable portion coupled with at least one fastener, wherein the rotatable portion rotates about the at least one fastener.

2. The system of claim 1, further comprising:
an upper edge disposed across the top of the stationary portion; and
a lower edge disposed across the bottom of the stationary portion, wherein the rotatable portion fits substantially within a boundary formed by the upper edge and the lower edge.

3. The system of claim 2, wherein the upper edge extends onto at least one side of the stationary portion.

4. The system of claim 2, wherein the lower edge extends onto at least one side of the stationary portion.

5. The system of claim 2, wherein
the upper edge extends across the top of the stationary portion,
and wherein the lower edge extends across the bottom of the stationary portion such that the upper edge and the lower edge form at least one gap on at least one side of the stationary portion.

6. The system of claim 5, wherein the rotatable portion is disposed such that it fits within the upper edge and the lower edge of the stationary portion.

7. The system of claim 6, the rotatable portion further comprising:
at least one grip, the at least one grip configured to fit within the at least one gap on the at least one side of the stationary portion.

8. The system of claim 6, wherein the upper edge and the lower edge form at least one gap on at least two sides of the stationary portion.

9. The system of claim 8, the rotatable portion further comprising:
at least one grip, the at least one grip configured to fit within at least one gap on the at least two sides of the stationary portion.

10. The system of claim 6, wherein an outer edge of the rotatable portion is substantially flush with the upper edge and lower edge of the stationary portion.

11. The system of claim 1, the stationary portion further comprising:
at least one front portion; and
at least one rear portion.

12. The system of claim 11, wherein the at least one rear portion is curved such that it substantially follows the contour of at least one protective equipment.

13. The system of claim 11, wherein the at least one rear portion includes at least one adhesive surface.

14. The system of claim 1, the rotatable portion further comprising:
at least one front portion; and
at least one rear portion.

15. The system of claim 1, wherein the at least one fastener includes at least one spring element.

16. A method of holding eyewear in place, the method including:
coupling a system for holding eyewear to at least one protective headgear;
disposing at least one strap of at least one eyewear around the at least one headgear;
lifting at least one rotatable portion of the system for holding eyewear; and
disposing an upper edge of the at least one eyewear below the at least one rotatable portion of the system such that the eyewear exerts an upward force on the at least one rotatable portion.
